(19) United States
(12) Patent Application Publication
(10) Pub. No.: US 2014/0020701 A1
(43) Pub. Date: Jan. 23, 2014

(54) Effervescent Composition and Method of Use

(71) Applicant: Alyson Galderisi, Cedar Grove, NJ (US)

(72) Inventor: Alyson Galderisi, Cedar Grove, NJ (US)

(21) Appl. No.: 13/412,453

(22) Filed: Mar. 5, 2012

(51) Int. Cl. A61K 8/97; A61K 8/49; A61K 8/365; A61K 8/92; A61K 8/36; A61K 8/66; A61K 8/67; A61Q 19/00; A61K 8/362; A61K 8/368

(52) U.S. Cl. 132/200; 424/43

(57) ABSTRACT

There is an anhydrous topical composition which, when spread on a topical surface and then sprayed with water, becomes effervescent and distributes the topical composition evenly over the surface. The composition has: a) a base compound of a carbonate or bicarbonate salt of an alkali metal or an alkaline earth metal; b) an acid compound; and c) an anhydrous carrier. The base compound and the acid compound are reactable and are present in amounts sufficient to cause the topical composition in suspension to effervesce when the topical composition is exposed to water. The effervescence acts to evenly distribute squalane over the topical surface upon which the composition was initially applied and provides a cosmetic effect if the topical surface is skin.

### BACKGROUND OF THE INVENTION

The present invention relates to an anhydrous topical composition that effervesces upon contact with water. The present invention further relates to a facial mask composition that effervesces upon contact with water.

Typical cleanser formulations are designed to effectively and efficiently remove previously applied face powder, rouge, foundation bases, eyeshadow and lipstick. Surfactants are also particularly useful in removing waterproof makeup. In addition, cleansers remove dirt and oil that accumulate on the skin or in the hair. To achieve this, commercial facial cleansers, usually in the form of a gel, lotion or cream, use surfactant ingredients. Further, a refreshing feeling is experienced when the surfactant is in contact with water because it generates a bubbly foam. Unfortunately, many surfactants are drying to the skin and/or are irritating to user. Because they contain surfactants, many cleansers cannot be routinely used in or in some instances even around the eye area.

Surfactants can interfere with the natural protective lipid barrier of the skin. There are two sources of skin surface lipids making up this important barrier: the sebaceous glands and the epidermis. Lipids are a diverse croup of compounds, comprising triglycerides, diglycerides, ceramides, free fatty acids, wax esters, cholesterol and cholesterol esters, and squalene. The quantity and composition of the skin surface lipids differ from place to place on the body, and may to some extent be related to the number of sebaceous glands in a given area of the skin. The condition of the skin surface lipids may also be affected by an essential fatty acid deficiency. Additionally, the lipid barrier can be diminished by exposure to harsh detergents or soaps containing surfactants. Therefore, cleansers are desired which do not interfere with the lipid barrier of the skin, but which clean thoroughly and, feel cool and refreshing.

Another desirable characteristic of cleansing products is the refreshing feeling derived from a cooling sensation on the skin. To achieve a physiological cooling effect on the skin, it is known in the prior art to use, for example, menthol or other similar low volatile compounds. Other compositions used include acyclic secondary and tertiary sulphoxides and sulphones, p-menthane and its derivatives. However, these compositions have a strong odor and are not desirable for use on the skin. Therefore, a less traditional form is needed that will provide a refreshing and cooling feeling that is pleasant to use.

Consumers of cleansers not only desire a product that cleans deeply, they desire a product that “feels” like it cleans deeply. This feeling is achieved when a cleanser product is cool, refreshing and tingly or bubbly during its use. The present invention provides a self-foaming system that produces a cooling sensation, cleans deeply, and “feels” like it cleans deeply but does not interfere with the delicate lipid barrier of the skin.

### SUMMARY OF THE INVENTION

The present invention relates to a cosmetic composition for application to a topical surface, such as the skin. The composition comprises an acid component, an alkali metal bicarbonate component and an anhydrous carrier. These two components and carrier may be mixed together in any order. Preferably, the acid and base components are first mixed together, and are then mixed with a cosmetically or pharmaceutically acceptable carrier. To use the system, the components are mixed and applied to the surface to be treated, such as the skin. Water is then applied to the composition which causes it to effervesce.

When the components are combined as described above, they produce carbon dioxide and generate an effervescent cleansing experience. The present invention includes a method of cooling the skin which comprises applying an effective amount of the cosmetic or pharmaceutical self-foaming system.

This effervescing composition and method is advantageous because it is gentler to the lipid barrier of the skin than a traditional cleanser containing a large percentage of surfactants, yet the system cleans deeply and has a tingly, bubbly and cool feel when used. The composition penetrates through the outermost “horny” layer of the epidermis when applied to skin, thus it helps prevent the development of dry skin and other undesirable results that can occur when using, for example, a cleanser with a large amount of surfactants.

An object of the present invention is to provide an anhydrous topical composition that effervesces upon contact with water. Another object of the present invention is to provide a method of dispersing squalane evenly over the topical surface onto which it has been applied. Still yet another object of the present invention is to provide a method for imparting a cosmetic effect to a topical surface of the human body. A still further object of the present invention is to provide a cooling and esthetically pleasing facial mask composition.

According to this and other objects of the invention, there is an anhydrous topical composition. The composition has: a) a base compound of a carbonate or bicarbonate salt of an alkali metal or an alkaline earth metal; b) an acid compound; and c) a cosmetically acceptable anhydrous vehicle. The base compound and the acid compound are reactable and are present in amounts sufficient to effervesce the topical composition when the topical composition is exposed to water.

According to this and other objects of the invention, there is a method for imparting a cosmetic effect to a topical surface. The method has the steps of a) applying the anhydrous composition described above to the topical surface of the human body, b) contacting the anhydrous composition with water to allow it to effervesce to form a effervesceed topical composition; and c) removing the effervesced topical composition from the topical surface.

### DETAILED DESCRIPTION OF THE INVENTION

Effervescence is a widely used, desirable and useful property of many commercial products that makes a product feel tingly or bubbly. Typically, the effervescent property of these products is based on the generation of carbon dioxide gas. The creation of carbon dioxide gas involves reacting an acid with bicarbonate. Bicarbonate is a relatively common household product also known as baking soda. In aqueous solution, bicarbonate releases carbon dioxide gas unless it is stabilized. Generally, this reaction proceeds rapidly because the acid and the alkaline neutralize each other. As the reaction progresses toward completion the effervescence diminishes and eventually ends when the reaction is complete.

It is known in the prior art to use sodium bicarbonate in personal care products. For example, a sodium bicarbonate effervescent system is used in a toothpaste product after overcoming challenges to make a product that is acceptable to the user. U.S. Pat. No. 4,487,757, incorporated herein by reference, discloses how to make a toothpaste container containing a baking soda toothpaste that overcomes the challenges of water solubility, salty taste, stability in the container, and its appearance when dispersed. An example of a two component foaming system is disclosed in U.S. Pat. No. 5,455,035 which provides for a surface active material in a cosmetic composition by neutralizing an organic acid surfactant precursor having 10 to 20 carbon atoms, and an alkaline neutralizing agent to provide the surface active material.

An important feature of the present invention is its ability to provide a effervescing system that is substantially non-irritating. In contrast to other foaming systems, which require the use of surfactants or other irritating agents to achieve the desired foaming property, the present invention achieves this by carbon dioxide generation in situ upon application.

The acid compound employed in the topical composition can be any organic or inorganic acid suitable for cosmetic and/or topical use. Organic acids are preferred. Suitable organic acids include, but are not limited to, group consisting of citric acid, tartaric acid, ascorbic acid, malic acid, maleic acid glutamic acid, glycolic acid, salicylic acid, cinnamic acid, nicotinic acid, cinnamomun cassia leaf extract, glycolic acid, papain, pyrus malus (apple) fruit extract, retinol, niacinamide, mixed fruit acids, botanical acids, ascorbic acid, asiaticoside, madecassic acid, saccharum officinarum, salix alba (willow) bark extract, vaccinium myrtillis (fruit/leaf extract), polyporous umbellatus (mushroom) extract, citrus aurantium dulcis (orange) fruit extract, citrus medica limonum (lemon) fruit extract, acer saccaharum (sugar maple) extract and combinations thereof. Citric acid is preferred.

The base compound employed in the topical composition is a carbonate or bicarbonate salt of an alkali metal or an alkaline earth metal. Suitable carbonate/bicarbonate compounds include, but are not limited to, sodium carbonate, sodium bicarbonate, potassium carbonate, potassium bicarbonate, magnesium carbonate, magnesium bicarbonate, calcium carbonate, calcium bicarbonate, and combinations thereof.

When mixed with water, bicarbonates and carbonates form carbonic acid and salts. The carbonic acid is unstable in water and breaks down rapidly to form CO₂ and water, per the following scheme:

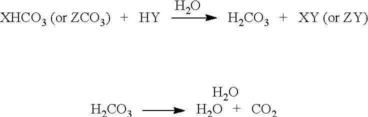

wherein

X is an alkali metal cation or NH₄⁺,

HY is a mineral or organic acid, and

XY is alkali or alkaline earth metal salt.

The acid compound and the base compound may be employed in even or uneven stoichiometric proportions. If desired, the two compounds may be employed in uneven stoichiometric proportions to ensure that one compound will be substantially consumed (converted) during the reaction. The base compound will be present in excess to ensure substantially complete reaction of the acid compound. However, it is also possible to formulate the products so that the acid compound is in excess. This will result in having unreacted acid following effervesceing. Accordingly, the composition following the addition of water, i.e., on the body of the consumer, will have a somewhat acid pH, which would exhibit a mild exfoliating effect on the skin. Such pH is preferably from about 2.0 to about 6.0. When the product compositions will be formulated to provide an acidic in use end product pH value, the formal ratio (equivalents acid/equivalents base) is most preferably from about 3:1 to about 1:1.

The acid compound and the base compound are present in amounts sufficient to effervesce the anhydrous when the anhydrous composition is exposed to water. The acid compound is preferably present at about 1 to about 30 wt % and most preferably present at about 3 to about 20 wt % based on the total weight of the topical composition. The base compound is preferably present at about 1 to about 30 wt % and most preferably present at about 3 to about 20 wt % based on the total weight of the topical composition.

The relative amounts of acid and base present in the composition will vary depending on the identity of each and their valances. Thus, when citric acid is used, which has three carboxyl groups available for reaction, the composition contains, for theoretical complete reaction, a molar amount that is one third the molar amount of sodium bicarbonate.

The anhydrous composition has an active ingredient in an amount sufficient to impart a cosmetic effect to the topical surface. The amount will vary considerably depending on the type of cosmetic effect desired and the properties of the active ingredient. The active ingredient will typically be present at about 0.001 to about 30 wt % and more typically present at about 0.01 to about 15 wt %, and most typically present at about 0.1 to about 5 wt % based on the total weight of the topical composition.

The present application may have one or more colorants/pigments as desired to modify the appearance of the anhydrous composition (initial) or the effervesceed composition (final). Examples of useful pigments/colorants include, but are not limited to, mica, iron oxides. Other color additives resulting from the carefully controlled heat treatment of food-grade carbohydrates may be used.

Useful cosmetically acceptable vehicles include non-aqueous fluids, such as polyols, hydrocarbon oils, triglycerides, ethers, fatty alcohols, fatty acid fluids, and low molecular weight alcohol having 2 to 8 carbons such as isopropyl alcohol. Preferably, the cosmetically acceptable vehicle is squalane.

It is possible for an ingredient to serve more than one function in the anhydrous composition. Squalane, glycerol or propylene can serve both as humectants and as vehicles.

The anhydrous topical composition takes a liquid or semisolid form. The composition is fluidic enough to be spread by hand, applicator, or implement. Useful forms include solution, lotion, paste, cream, gel, and pomade. Composition fluid properties can be modified as desired by the addition of adjuvants such as thickeners, viscosity modifiers, and the like.

The anhydrous composition can be applied to any topical surface, exposed to water, and then removed by wiping the surface. Useful topical surfaces include skin. Any area of the skin, such as the hands and face, may be treated.

The anhydrous composition is useful in any type of cosmetic product. Suitable compositions include facial mask, skin cleanser, and skin exfoliant. A preferred composition is a facial mask composition.

Facial mask compositions can be spread over the surface of the skin of the face. Then water is applied to the face. Water may be dripped on the face, splashed, or applied by means of a wet towelette. Preferably, water is sprayed onto the face. The water causes the acid compound and the base compound to react to form carbon dioxide and a neutralized salt. The evolution of carbon dioxide causes the facial mask composition to effervesce. After the effervescing stops, typically after about 5 to 10 minutes. This gives time for the squalane or another humectants to impart a cosmetic effect(s) or benefit(s) or to permit cleansing of skin pores. The clear mask composition is then removed, preferably by wiping.

Squalane is most preferably included in the anhydrous liquid as it penetrates through the uppermost or “horny” layer of the skin. This enables any antioxidant to penetrate and impart a cosmetic effect. Further, when the effervescence has stopped and the surface is wiped with a dry cloth, sufficient squalane remains on the surface to act as a humectant.

This composition includes no clay material, and when the effervescing has stopped, all that remains on the skin is a clear, refreshing liquid. Wiping away the majority of this liquid is preferred as this method of removal leaves behind a thin layer of squalane or other humectant used.

The composition preferably exhibits a thixotropic rheology. The composition has a viscosity sufficient to permit application to the body of the consumer and which avoids dripping or running from the body regardless of spatial orientation. Typically, the composition has a viscosity of about 500 to 1,000,000 cps. The composition preferably exhibits a viscosity of about 10,000 to about 500,000 cps at 25.degree. C., and most preferably from about 10,000 to 200,000 cps, as measured by a Brookfield viscometer having an appropriate spindle for the viscosity region. This viscosity level of the thixotropic composition allows the user to apply the composition to the face or other body surface without having the composition drip off. Additionally, this viscosity level is beneficial in that it prevents excessive dripping of the wetted composition. This viscosity level also does not prevent reaction of the acid compound and the base compound.

The composition may be stored in any suitable container. Preferably, however, the composition is sold in an airtight, single-use pouch to prevent moisture from prematurely causing reaction. As previously discussed, a small amount of a deliquescent material can be incorporated in the composition to absorb any moisture that may be present. Suitable materials are zinc chloride, calcium chloride, and silica.

The composition may be in the form of an anhydrous gel having a thixotropic rheology. The composition is anhydrous and must be kept dry until use. Alternatively, the carrier, acid and base are stored separately to prevent interaction. Keeping these ingredients separate also allows a very pure compound to be used on a topical surface, not requiring preservatives or drying agents.

Mixing the components of the composition can be done in any order. The composition can be mixed immediately before being applied to a topical surface or it may be pre-mixed and applied later as long as the mixture is maintained free from water.

In one embodiment, however, the system is used primarily for its cooling effects, and not as a cleanser. For example, the system can be advantageously used as the base of a foundation or sun-care product. These types of products, when applied in hot and/or humid weather conditions, can feel uncomfortable on the skin. The application of the system under such conditions will provide a cooling effect, in addition to the other intended effect of the product and avoid some of the discomfort often associated with more traditional products of this type.

In a particularly preferred embodiment, however, the system is used for removing dirt, oil, makeup, and other impurities from the skin. As already noted, because of the ability to effervesce by means of carbon dioxide gas generation, the effects of the composition are mild and do not interfere with the natural protective lipid barrier of the skin. In addition, carbon dioxide generation augments the cooling and cleansing properties of the system.

The present invention also includes a method of producing the cooling sensation when an effective amount of the components of the system are dispensed and applied to the skin or the hair. When combined with appropriate additional components, the resulting self-foaming systems successfully cool skin surfaces, including around the eyes and lips. Moreover, they clean the skin and remove transfer-resistant cosmetics. All this is achieved while remaining gentle and substantially non-irritating to the user.

It was surprisingly found that there is an anhydrous topical composition that effervesces when contacted with water and that creates a cooling sensation. It was still further surprisingly found that there is an composition that both effervesces and evenly disperses squalane over the surface of the topical surface onto which it is applied.

During and after the effervescence, the surface is cooled, which helps to refresh the topical surface of the human body to which the composition is applied. The phase change is aesthetically appealing to the product user and signals the end of the application/use cycle of the composition.

The topical composition is substantially anhydrous in that the substantial absence of water is critical to prevent premature reaction of the acid compound and the base compound. Prevention of premature reaction prevents effervescence. To prevent premature reaction and loss of reactants, the compositions of the present invention may also contain a small amount of a deliquescent agent, for example, calcium chloride, fumed silica or zinc chloride. Typically, the amount of deliquescent agent is about 2% or less by total weight of the composition.

As referred to herein, the term “topical surface” refers to the exterior surface of the epidermis (the skin). Topical surface includes the exterior surface of any portion of the human body.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

1. A composition resulting from combining, comprising:
a) a base compound of a carbonate or bicarbonate salt of an alkali metal or an alkaline earth metal;
b) an anhydrous acid compound; and
c) an anhydrous carrier;
wherein the base compound and the acid compound are reactable and are present when combined in amounts sufficient to effervesce when the composition is exposed to water.

2. The composition of claim 1, wherein the base compound and acid compound are present at a weight ratio of about 1:1 to about 1:2.

3. The composition of claim 1, wherein the acid compound and the base compound are combined to create a mixed acid/base powder.

4. The composition of claim 3, wherein the mixed acid/base powder and the anhydrous carrier are present at a volume ratio of about 1:1.

5. The composition of claim 1, wherein the anhydrous carrier is squalane, glycerin, lanolin, Vitamin E, Vitamin A and/or olive oil.

6. The composition of claim 1, wherein the base compound is selected from the group consisting of sodium carbonate, sodium bicarbonate, potassium carbonate, potassium bicarbonate, magnesium carbonate, magnesium bicarbonate, calcium carbonate, calcium bicarbonate and combinations thereof.

7. The composition of claim 1, wherein the acid compound is selected from the group consisting of citric acid, tartaric acid, ascorbic acid, malic acid, maleic acid, glutamic acid, glycolic acid, salicylic acid, cinnamic acid, nicotinic acid, cinnamomun cassia leaf extract, glycolic acid, papain, pyrus malus (apple) fruit extract, retinol, niacinamide, mixed fruit acids, botanical acids, ascorbic acid, asiaticoside, madecassic acid, saccharum officinarum, salix alba (willow) bark extract, vaccinium myrtillis (fruit/leaf extract), polyporous umbellatus (mushroom) extract, citrus aurantium dulcis (orange) fruit extract, citrus medica limonum (lemon) fruit extract, acer saccaharum (sugar maple) extract and combinations thereof.

8. The composition of claim 1, wherein the composition is a facial mask composition.

9. A method for imparting a cosmetic effect to a topical surface of a human body, comprising the steps of:
(a) applying to the topical surface
i) a base compound of a carbonate or bicarbonate salt of an alkali metal or an alkaline earth metal;
ii) an acid compound; and
iii) an anhydrous carrier compound;
wherein the base compound and the acid compound are reactable and are present when combined in amounts sufficient to effervesce when the anhydrous composition is sprayed with water;
(b) after step (a), contacting the applied compounds with water resulting in effervescence; and
(c) after step (b), removing the applied compound from the topical surface.

11. The method of claim 9, wherein the applied compound is not removed until it has stopped effervescing.

12. The method of claim 9, wherein the topical surface is a face.

13. The method of claim 9, wherein the pH of the composition following step (b) is from about 2 to about 6.

14. The method of claim 9, wherein the composition cools the topical surface when it effervesces.

15. The method of claim 9, wherein the applied compound is removed without water.

16. The method of claim 9, wherein the compounds in step (a) are applied in any order.
